(19) United States
(12) Patent Application Publication
(10) Pub. No.: US 2025/0169793 A1
(43) Pub. Date: May 29, 2025

(54) MEDICAL SYSTEM AND MEDICAL METHOD THEREOF

(71) Applicant: Industrial Technology Research Institute, Hsinchu (TW)

(72) Inventors: Shang-Chih Hung, Hsinchu County (TW); Ho-Hsin Lee, Hsinchu City (TW); Jian-Ren Chen, Hsinchu County (TW)

(73) Assignee: Industrial Technology Research Institute, Hsinchu (TW)

(21) Appl. No.: 18/963,789

(22) Filed: Nov. 29, 2024

Related U.S. Application Data
(60) Provisional application No. 63/603,641, filed on Nov. 29, 2023.

(30) Foreign Application Priority Data
Jul. 16, 2024 (TW) 113126546

(51) Int. Cl. A61B 8/00

(52) U.S. Cl. CPC A61B 8/4254; A61B 8/4218; A61B 8/54; A61B 8/565

(57) ABSTRACT

A medical system and a medical method thereof. The medical method includes following steps: the sensing data acquisition sub-module obtains sensing data between the mechanical arm and a patient body surface through the sensor; the mechanical arm adjustment sub-module uses the sensing data to adjust the mechanical arm; and the simulated force feedback calculation sub-module uses the sensing data to obtain a force feedback value, and the simulated force feedback calculation sub-module uses the force feedback value to trigger the force feedback manual controller.

### CROSS-REFERENCE TO RELATED APPLICATION

This application claims the priority benefit of U.S. provisional application Ser. No. 63/603,641, filed on Nov. 29, 2023 and Taiwan application serial no. 113126546, filed on Jul. 16, 2024. The entirety of each of the above-mentioned patent applications is hereby incorporated by reference herein and made a part of this specification.

### TECHNICAL FIELD

The present disclosure relates to a medical system and a medical method thereof.

### BACKGROUND

Currently, when a physician performs a remotely controlled ultrasound examination, the physician must rely on the patient side camera to provide real-time images to confirm the position of the ultrasound probe on the patient body surface, and the physician also needs to operate the force feedback manual controller to adjust the ultrasound probe. Since the patient side camera usually only provides patient images from a specific angle, the physician needs to check back and forth between the patient image display device and the patient ultrasound image display device to confirm the position of the ultrasound probe on the patient body surface.

In addition, since the physician does not personally hold the ultrasound probe, the physician lacks the operating experience of pressing on the patient body surface on site.

### SUMMARY

The medical system of the present disclosure includes a patient side device and a physician side device. The patient side device includes a storage medium, a processor, a probe holder and a mechanical arm, wherein the storage medium stores a plurality of sub-modules, and the plurality of sub-modules include a sensing data acquisition sub-module, a mechanical arm adjustment sub-module and a simulated force feedback calculation sub-module, wherein the processor is coupled to the storage medium, the probe holder and the mechanical arm and accesses and executes the plurality of sub-modules, wherein the probe holder includes a sensor. The physician side device is communicatively connected to the patient side device, wherein the physician side device includes a force feedback manual controller, wherein the sensing data acquisition sub-module obtains sensing data between the mechanical arm and a patient body surface through the sensor; the mechanical arm adjustment sub-module uses the sensing data to adjust the mechanical arm; the simulated force feedback calculation sub-module uses the sensing data to obtain a force feedback value, and the simulated force feedback calculation sub-module uses the force feedback value to trigger the force feedback manual controller.

The medical method of the present disclosure includes following steps: the sensing data acquisition sub-module obtains sensing data between the mechanical arm and a patient body surface through the sensor; the mechanical arm adjustment sub-module uses the sensing data to adjust the mechanical arm; and the simulated force feedback calculation sub-module uses the sensing data to obtain a force feedback value, and the simulated force feedback calculation sub-module uses the force feedback value to trigger the force feedback manual controller.

Based on the above, the medical system and medical method thereof of the present disclosure can use the sensing data to adjust the mechanical arm after obtaining the sensing data between the mechanical arm and the patient body surface. In this way, the physician does not need to check back and forth between the patient image display device and the patient ultrasound image display device to confirm the position of the ultrasound probe on the patient body surface. Furthermore, the medical system and medical method thereof of the present disclosure can also use the sensing data to obtain the force feedback value, and use the force feedback value to trigger the force feedback manual controller of the physician side device. Based on this, the physician will be able to obtain a simulated feeling of pressing on the patient body surface.

### BRIEF DESCRIPTION OF THE DRAWINGS

FIG. 1 is a schematic diagram of a medical system according to an embodiment of the present disclosure.

FIG. 2 is a flow chart of a medical method according to an embodiment of the present disclosure.

FIG. 3 is an implementation example of step S210 and step S230 shown in FIG. 2.

FIG. 4 is an implementation example of step S230 shown in FIG. 2.

FIG. 5 is an implementation example of step S210 and step S250 shown in FIG. 2.

FIGS. 6, 7 and 8 are further explanations of FIG. 5.

### DESCRIPTION OF THE EMBODIMENTS

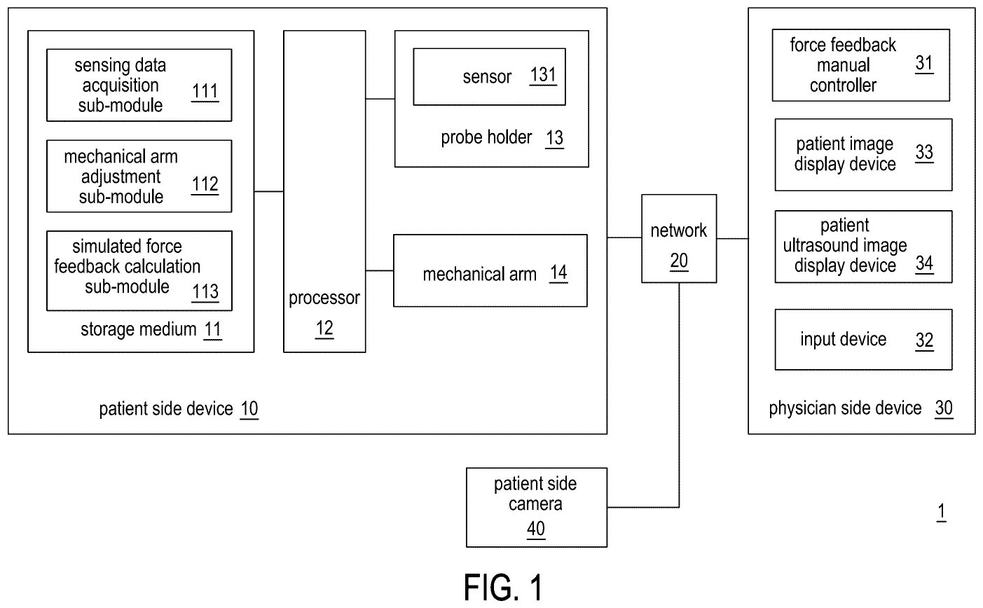

FIG. 1 is a schematic diagram of a medical system 1 according to an embodiment of the present disclosure. Please refer to FIG. 1. The medical system 1 can include a patient side device 10 and a physician side device 30. The patient side device 10 can include a storage medium 11, a processor 12, a probe holder 13 and a mechanical arm 14. The physician side device 30 can be communicatively connected to the patient side device 10 (via the network 20). In one embodiment, the patient side device 10 and the physician side device 30 can be installed at different locations. In other words, the physician does not have direct contact with the patient.

The storage medium 11 is, for example, any type of fixed or removable random access memory (random access memory, RAM), read-only memory (read-only memory, ROM), flash memory, hard disk drive (HDD), solid state drive (SSD) or similar components or a combination of the above components, used to store plurality of modules or various applications that can be executed by the processor 12. In this embodiment, the storage medium 11 can store a plurality of sub-modules, and the plurality of sub-modules can include a sensing data acquisition sub-module 111, a mechanical arm adjustment sub-module 112, and a simulated force feedback calculation sub-module 113.

The processor 12 is, for example, a central processing unit (CPU), or other programmable general-purpose or special-purpose micro control unit (MCU), microprocessor, digital signal processor (DSP), programmable controller, application specific integrated circuit (ASIC), graphics processing unit (GPU), image signal processor (ISP), image processing unit (IPU), arithmetic logic unit (ALU), complex programmable logic device (CPLD), field programmable gate array (FPGA) or others Similar elements or combinations of the above elements. The processor 12 can be coupled to the storage medium 11, the probe holder 13 and the mechanical arm 14 and access and execute the plurality of sub-modules.

Probe holder 13 can include sensor 131. In one embodiment, the probe holder 13 can be used to fix the ultrasonic probe. In one embodiment, the sensor 131 is, for example, an IoT (Internet of Things) sensing element such as ToFs (Time of Flight), Load Cell, and/or Limit Switch, but the disclosure is not limited thereto.

The physician side device 30 can include a force feedback manual controller 31. The force feedback manual controller 31 is, for example, a 3D joystick. In one embodiment, the physician side device 30 can include an input device 32. Furthermore, the physician side device 30 can also include a patient image display device 33 and a patient ultrasound image display device 34.

The patient side camera 40 can be connected to the physician side device 30 via network 20.

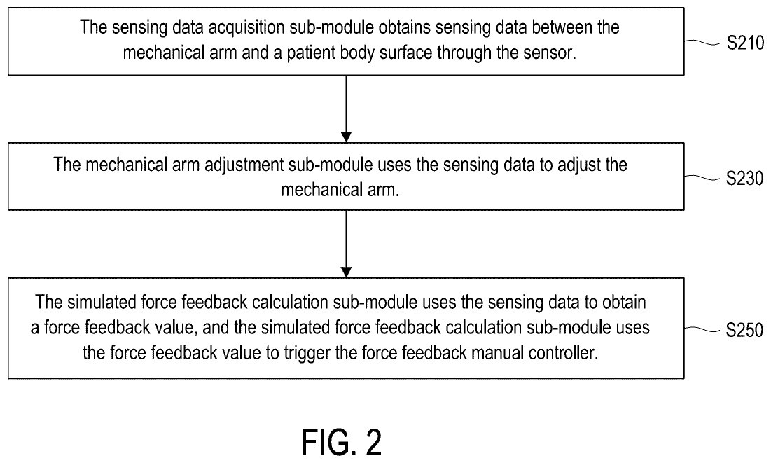

FIG. 2 is a flow chart of a medical method according to an embodiment of the present disclosure, wherein the medical method can be implemented by the medical system 1 shown in FIG. 1. Please refer to both FIG. 1 and FIG. 2.

In step S210, the sensing data acquisition sub-module 111 can obtain sensing data between the mechanical arm 14 and a patient body surface through the sensor 131.

In step S230, the mechanical arm adjustment sub-module 112 can use the sensing data to adjust the mechanical arm 14.

In step S250, the simulated force feedback calculation sub-module 113 can use the sensing data to obtain a force feedback value, and the simulated force feedback calculation sub-module 113 can use the force feedback value to trigger the force feedback manual controller 31.

It is worth first mentioning here that although the patient side camera 40 can usually only provide patient images from a specific angle, through the present disclosure, the physician does not need to check back and forth between the patient image display device 33 and the patient ultrasound image display device 34 to confirm the position of the ultrasound probe on the patient body surface, and the physician does not need to frequently adjust the force feedback manual controller 31 to move the probe holder 13. Specifically, through the present disclosure, the physician will be able to focus on interpreting the ultrasound image displayed by the patient ultrasound image display device 34, and the physician will be able to obtain a simulated operating experience of pressing on the patient body surface. Implementation examples of the above step S210, step S230 and step S250 will be further described below.

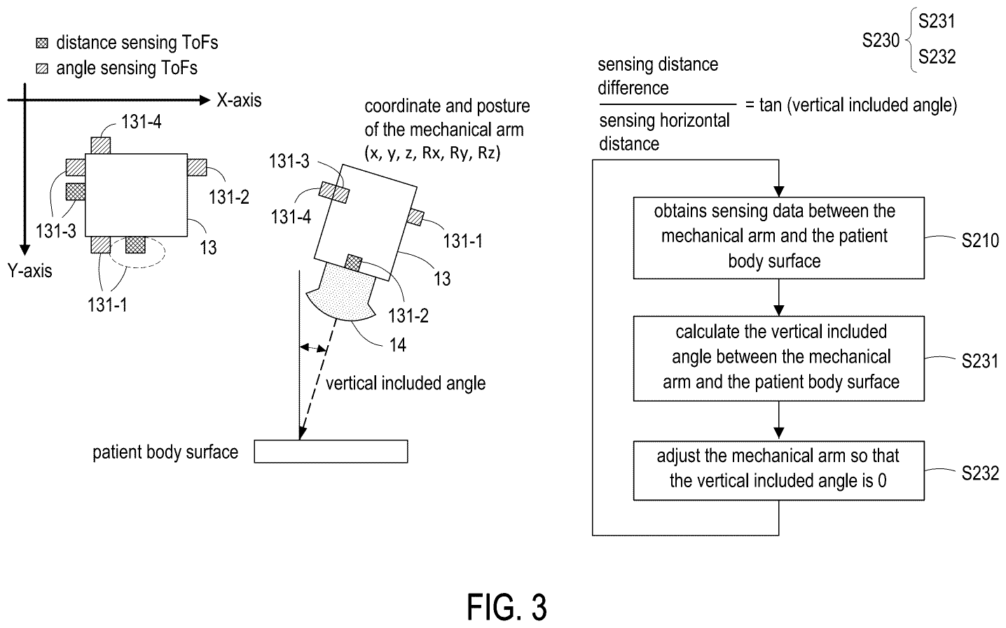

FIG. 3 is an implementation example of step S210 and step S230 shown in FIG. 2. Please refer to FIG. 1, FIG. 2 and FIG. 3 at the same time. As shown in FIG. 3, the sensor 131 can include a first sensor 131-1, a second sensor 131-2, a third sensor 131-3 and a fourth sensor 131-4. In this embodiment, the first sensor 131-1, the second sensor 131-2, the third sensor 131-3 and the fourth sensor 131-4 can be respectively installed at the front, rear, left and right corners/end points of the probe holder 13. In one embodiment, the first sensor 131-1, the second sensor 131-2, the third sensor 131-3 and the fourth sensor 131-4 are, for example, distance sensing ToFs (Time of Flight) and/or angle sensing ToFs. It should be noted here that the present disclosure does not limit the amount of the sensor 131.

Please continue to refer to FIG. 3. In this embodiment, the sensing data can include a sensing distance difference between the mechanical arm 14 and the patient body surface, and the sensing data can include a sensing horizontal distance between the mechanical arm 14 and the patient body surface. In other words, in step S210 shown in FIG. 3, the sensing data acquisition sub-module 111 can obtain sensing distance difference between the mechanical arm 14 and the patient body surface through the first sensor 131-1, the second sensor 131-2, the third sensor 131-3 and the fourth sensor 131-4, and the sensing data acquisition sub-module 111 can obtain the sensing horizontal distance between the mechanical arm 14 and the patient body surface through the first sensor 131-1, the second sensor 131-2, the third sensor 131-3 and the fourth sensor 131-4. Next, in step S231, the mechanical arm adjustment sub-module 112 can calculate the vertical included angle using the sensing distance difference and the sensing horizontal distance. Then, the mechanical arm adjustment sub-module 112 can use the vertical included angle to adjust the mechanical arm 14. Specifically, in step S232, the mechanical arm adjustment sub-module 112 can adjust the mechanical arm 14 so that the vertical included angle is 0. Thereby, the mechanical arm 14 can be made as perpendicular to the patient body surface as possible, so that the physician can obtain clearer ultrasound images through the patient ultrasound image display device 34.

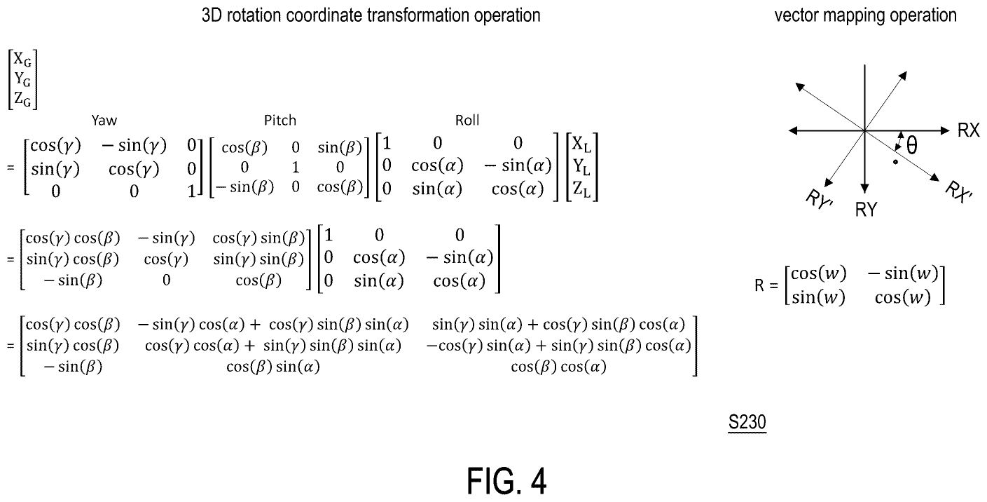

FIG. 4 is an implementation example of step S230 shown in FIG. 2. Please refer to FIG. 1, FIG. 2 and FIG. 4 at the same time. In this embodiment, mechanical arm 14 can correspond to a mechanical arm posture (ie, Rx, Ry, Rz). The mechanical arm adjustment sub-module 112 can perform a translation operation to translate a moving reference point from the mechanical arm 14 to the probe holder 13. Then, the mechanical arm adjustment sub-module 112 can use the mechanical arm posture, a 3D rotation coordinate transformation operation, and a vector mapping operation to calculate a movement distance and movement component corresponding to the probe holder 13. In other words, the mechanical arm adjustment sub-module 112 can convert the sensing data (such as the above-mentioned sensing distance difference) through coordinate conversion to calculate the amount that the mechanical arm 14 needs to move/rotate. In particular, since the direction and the coordinate system of the force feedback manual controller 31 are different from those of the mechanical arm 14, in order to allow the physician to feel that the movements of the mechanical arm 14 are consistent with the force feedback manual controller 31, the medical system 1 of the present disclosure can perform the 3D rotation coordinate transformation operation and the vector mapping operation.

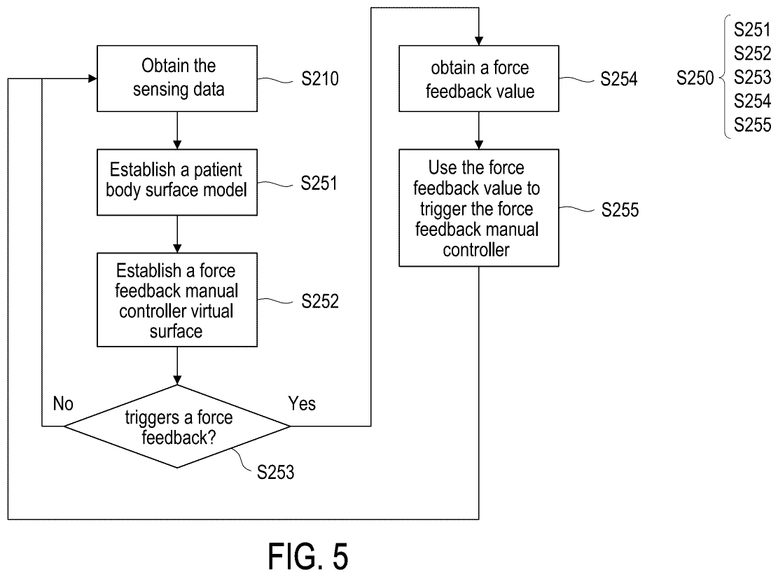

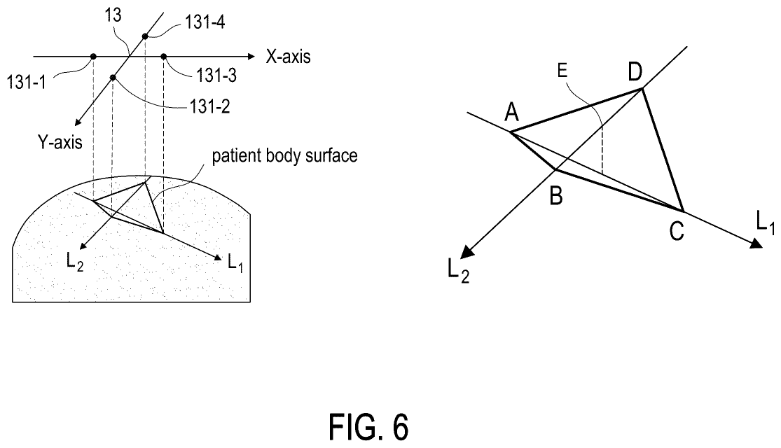

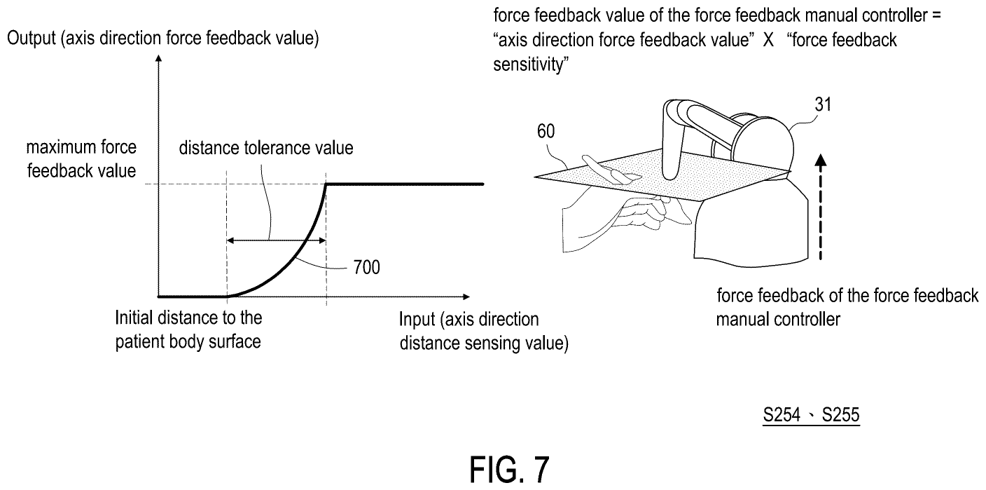

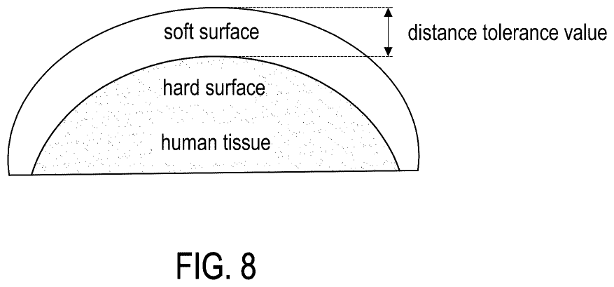

FIG. 5 is an implementation example of step S210 and step S250 shown in FIG. 2. FIGS. 6, 7 and 8 are further explanations of FIG. 5. Please refer to FIG. 1, FIG. 2, FIG. 5, FIG. 6, FIG. 7 and FIG. 8 at the same time. In this embodiment, the mechanical arm 14 can correspond to the mechanical arm posture, and the probe holder 13 can correspond to a probe coordinate. The sensing data can include mechanical arm posture and the probe coordinate, and the sensing data can include a pressure value and a deformation amount of the mechanical arm 14 pressing down on the patient body surface.

Please continue to refer to FIG. 5. In step S210, the sensing data acquisition sub-module 111 can obtain the sensing data between the mechanical arm 14 and the patient body surface through the sensor 131. In step S251, the simulated force feedback calculation sub-module 113 can use the sensing data to establish a patient body surface model. Next, in step S252, the simulated force feedback calculation sub-module 113 can use the patient body surface model to establish a force feedback manual controller virtual surface corresponding to the force feedback manual controller 31.

Specifically, as shown in FIG. 6, it is assumed that the projection points of the first sensor 131-1, the second sensor 131-2, the third sensor 131-3 and the fourth sensor 131-4 on the patient body surface are point A, point B, point C and point D, respectively, and assume that the projection point of probe holder 13 on the patient body surface is point E. The simulated force feedback calculation sub-module 113 can use the sensing distance difference, the probe coordinate, and the mechanical arm posture to calculate the coordinates of point A, point B, point C, point D and point E to establish the patient body surface model. Furthermore, since the axis L₁ and the axis L₂ may not intersect in the 3D space, the simulated force feedback calculation sub-module 113 can divide the patient body surface into four sub-planes to form a tetrahedral patient body surface model. Then, the simulated force feedback calculation sub-module 113 can simulate/project the patient body surface model into a virtual surface of the side of the force feedback manual controller 31.

In one embodiment, the patient body surface can include a soft surface, wherein the thickness of the soft surface can indicate a distance tolerance value, wherein the force feedback value can be associated with the distance tolerance value.

Please refer to FIG. 5. In step S253, the simulated force feedback calculation sub-module 113 can determine whether the force feedback manual controller 31 triggers a force feedback. If the simulated force feedback calculation sub-module 113 determines that the force feedback manual controller 31 has triggered force feedback (the judgment result of step S253 is “yes”), then in step S254, the simulated force feedback calculation sub-module 113 can obtain a force feedback value. Next, in step S255, the simulated force feedback calculation sub-module 113 can use the force feedback value to trigger the force feedback manual controller 31.

In more detail, as shown in FIGS. 7 and 8, it is assumed that the simulated force feedback calculation sub-module 113 has established the force feedback manual controller virtual surface 60. The simulated force feedback calculation sub-module 113 can obtain the force feedback value according to the nonlinear equation 700. It is worth mentioning here that since the patient body surface is not a hard steel surface, when the distance that the force feedback manual controller 31 presses down on the force feedback manual controller virtual surface 60 (i.e., the amount of pressing deformation) does not exceed the distance tolerance value, the force feedback value obtained by the simulated force feedback calculation sub-module 113 will be 0. In other words, the force feedback manual controller 31 can further press down on the patient body surface (the force feedback manual controller virtual surface 60) for a certain distance and the physician still will not feel the force feedback value through the force feedback manual controller 31. On the other hand, when the distance that the force feedback manual controller 31 presses down on the force feedback manual controller virtual surface 60 exceeds the distance tolerance value, the simulated force feedback calculation sub-module 113 can obtain the force feedback value according to the nonlinear equation 700.

In one embodiment, the input device 32 can receive an adjustment operation to adjust the distance tolerance value. In detail, since different parts of the patient body have different softness, the physician can operate the input device 32 to adjust the distance tolerance value. Based on this, when the physician operates the physician side device 30 to observe different parts of the patient body, the simulated force feedback calculation sub-module 113 will obtain the force feedback value based on the different distance tolerance values (also known as “force feedback sensitivity”).

In other embodiments, in addition to the distance tolerance value, the force feedback value can be associated with the following parameters:

(1) Slope: the ratio of the force feedback value from the patient body surface to the hard surface and the distance.

(2) Error: error of the above-mentioned sensing distance difference and/or the sensing horizontal distance.

(3) Ratio: the ratio of the sensing distance difference (mm) converted into the force feedback value (g).

Similarly, the physician can operate the input device 32 to adjust the above (1), (2) and (3) to make the force feedback value more approximate to the softness of the real patient body.

To sum up, the medical system and medical method thereof of the present disclosure can use the sensing data to adjust the mechanical arm after obtaining the sensing data between the mechanical arm and the patient body surface. In this way, the physician does not need to check back and forth between the patient image display device and the patient ultrasound image display device to confirm the position of the ultrasound probe on the patient body surface. Furthermore, the medical system and medical method thereof of the present disclosure can also use the sensing data to obtain the force feedback value, and use the force feedback value to trigger the force feedback manual controller of the physician side device. Based on this, the physician will be able to obtain a simulated feeling of pressing on the patient body surface.

What is claimed is:

1. A medical system, including:
a patient side device, including a storage medium, a processor, a probe holder and a mechanical arm, wherein the storage medium stores a plurality of sub-modules, and the plurality of sub-modules include a sensing data acquisition sub-module, a mechanical arm adjustment sub-module and a simulated force feedback calculation sub-module, wherein the processor is coupled to the storage medium, the probe holder and the mechanical arm and accesses and executes the plurality of sub-modules, wherein the probe holder includes a sensor; and
a physician side device, communicatively connected to the patient side device, wherein the physician side device includes a force feedback manual controller, wherein
the sensing data acquisition sub-module obtains sensing data between the mechanical arm and a patient body surface through the sensor;
the mechanical arm adjustment sub-module uses the sensing data to adjust the mechanical arm;
the simulated force feedback calculation sub-module uses the sensing data to obtain a force feedback value, and the simulated force feedback calculation sub-module uses the force feedback value to trigger the force feedback manual controller.

2. The medical system of claim 1, wherein the sensing data includes a sensing distance difference between the mechanical arm and the patient body surface, and the sensing data includes a sensing horizontal distance between the mechanical arm and the patient body surface, wherein
the mechanical arm adjustment sub-module uses the sensing distance difference and the sensing horizontal distance to calculate a vertical included angle;
the mechanical arm adjustment sub-module uses the vertical included angle to adjust the mechanical arm.

3. The medical system of claim 1, wherein the mechanical arm corresponds to a mechanical arm posture, wherein
the mechanical arm adjustment sub-module performs a translation operation to translate a moving reference point from the mechanical arm to the probe holder;
the mechanical arm adjustment sub-module uses the mechanical arm posture, a 3D rotation coordinate transformation operation and a vector mapping operation to calculate a movement distance and a movement component corresponding to the probe holder.

4. The medical system of claim 1, wherein the mechanical arm corresponds to a mechanical arm posture, and the probe holder corresponds to a probe coordinate, wherein the sensing data includes the mechanical arm posture and the probe coordinate, and the sensing data includes a pressure value and a deformation amount of the mechanical arm pressing down on the patient body surface.

5. The medical system of claim 1, wherein
the simulated force feedback calculation sub-module uses the sensing data to establish a patient body surface model;
the simulated force feedback calculation sub-module uses the patient body surface model to establish a force feedback manual controller virtual surface corresponding to the force feedback manual controller.

6. The medical system of claim 1, wherein the patient body surface includes a soft surface, wherein a thickness of the soft surface indicates a distance tolerance value, wherein the force feedback value is associated with the distance tolerance value.

7. The medical system of claim 6, wherein the physician side device further includes an input device, wherein
the input device receives an adjustment operation to adjust the distance tolerance value.

8. A medical method, applicable to a medical system including a patient side device and a physician side device, wherein the patient device includes a storage medium, a processor, a probe holder and a mechanical arm, wherein the storage medium stores a plurality of sub-modules, and the plurality of sub-modules include a sensing data acquisition sub-module, a mechanical arm adjustment sub-module and a simulated force feedback calculation sub-module, wherein the probe holder includes a sensor, wherein the physician side device includes a force feedback manual controller, wherein the medical method include following steps:
the sensing data acquisition sub-module obtains sensing data between the mechanical arm and a patient body surface through the sensor;
the mechanical arm adjustment sub-module uses the sensing data to adjust the mechanical arm; and
the simulated force feedback calculation sub-module uses the sensing data to obtain a force feedback value, and the simulated force feedback calculation sub-module uses the force feedback value to trigger the force feedback manual controller.

9. The medical method of claim 8, wherein the sensing data includes a sensing distance difference between the mechanical arm and the patient body surface, and the sensing data includes a sensing horizontal distance between the mechanical arm and the patient body surface, wherein the mechanical arm adjustment sub-module uses the sensing data to adjust the mechanical arm includes:
the mechanical arm adjustment sub-module uses the sensing distance difference and the sensing horizontal distance to calculate the vertical included angle; and
the mechanical arm adjustment sub-module uses the vertical included angle to adjust the mechanical arm.

10. The medical method of claim 8, wherein the mechanical arm corresponds to a mechanical arm posture, wherein the mechanical arm adjustment sub-module uses the sensing data to adjust the mechanical arm includes:
the mechanical arm adjustment sub-module performs a translation operation to translate a moving reference point from the mechanical arm to the probe holder; and
the mechanical arm adjustment sub-module uses the mechanical arm posture, a 3D rotation coordinate transformation operation and a vector mapping operation to calculate a movement distance and a movement component corresponding to the probe holder.

11. The medical method of claim 8, wherein the mechanical arm corresponds to a mechanical arm posture, and the probe holder corresponds to a probe coordinate, wherein the sensing data includes the mechanical arm posture and the probe coordinate, and the sensing data includes a pressure value and a deformation amount of the mechanical arm pressing down on the patient body surface.

12. The medical method of claim 8, wherein the simulated force feedback calculation sub-module uses the sensing data to obtain the force feedback value, and the simulated force feedback calculation sub-module uses the force feedback value to trigger the force feedback manual controller includes:
the simulated force feedback calculation sub-module uses the sensing data to establish a patient body surface model; and
the simulated force feedback calculation sub-module uses the patient body surface model to establish a force feedback manual controller virtual surface corresponding to the force feedback manual controller.

13. The medical method of claim 8, wherein the patient body surface includes a soft surface, wherein a thickness of the soft surface indicates a distance tolerance value, wherein the force feedback value is associated with the distance tolerance value.

14. The medical method of claim 13, wherein the physician side device further includes an input device, and the medical method further includes following steps:
the input device receives an adjustment operation to adjust the distance tolerance value.
